(19) United States
(12) Patent Application Publication
(10) Pub. No.: US 2003/0135183 A1
(43) Pub. Date: Jul. 17, 2003

(54) Absorbent articles having stretchable chassis

(76) Inventors: Sunita Pargass, Tyrone, GA (US); Joseph B. Vergona, Suwanee, GA (US); Stacy Driskell, Alpharetta, GA (US)

Correspondence Address: Christopher C. Campbell, Esq., Hunton & Williams, Suite 1200, 1900 K Street, NW, Washington, DC, 20006-1109, US

(21) Appl. No.: 10/012,368

(22) Filed: Dec. 12, 2001

(51) Int. Cl. A61F 13/15

(52) U.S. Cl. 604/385.01

(57) ABSTRACT

An absorbent article having a chassis having a chassis layer with a chassis surface area, an elastic portion, a topsheet attached to the chassis, at least a portion of the topsheet being liquid permeable, and an absorbent material disposed between the topsheet and the chassis. The elastic portion has a first carrier layer with a first surface area, and an elastic layer attached to the first carrier layer and having an elastic layer surface area. The elastic layer is an elastic scrim.

### FIELD OF THE INVENTION

The present invention relates to absorbent articles. Specifically, the invention relates to absorbent articles having a chassis that can be stretched. The absorbent articles of the invention have improved fitting characteristics and are capable of being used for a number of different sized wearers.

### BACKGROUND OF THE INVENTION

Disposable absorbent articles, such as diapers, sanitary products, incontinent pads, and the like have obtained wide acceptance by consumers. Such absorbent articles generally include a liquid permeable topsheet, a liquid impermeable backsheet for preventing body exudates from leaking out, and an absorbent member interposed there between for absorbing such body exudates. Conventional liquid impermeable backsheets usually provide satisfactory liquid impermeability, but these sheets typically are not flexible enough to closely conform to the shape of wearer's body, and consequently provide poor leakage protection. They also limit the ability of the absorbent article to stretch.

Infants and other incontinent individuals wear absorbent articles such as diapers to receive and contain urine and other body exudates. Absorbent articles function both to contain discharged materials and to isolate the materials from the body of the wearer and from the wearer's garments and bed clothing. Disposable absorbent articles having many different basic designs are known to the art. For example, U.S. Reissue Pat. No. 26,152, describes a conventional disposable diaper which has achieved worldwide acceptance and commercial success. Further, U.S. Pat. No. 5,246,433 discloses a unitary disposable absorbent article that can be used as a training pant. The disclosures of these documents are incorporated by reference herein in their entirety.

In the market today, the consumer has a number of different basic diaper designs to choose from depending on the desired options, comfort and cost. These diaper designs include conventional diapers, belted diapers, and “pull-on” type diapers or training pants. However, many of the absorbent articles on the market today are capable of fitting only a small range of wearer sizes and therefore, the consumer must continually monitor the size of the wearer to determine which diaper will comfortably and effectively fit the wearer. Thus, the consumer must purchase different diapers depending on the desired characteristics for the intended use.

Conventional diaper designs generally are the least expensive type of absorbent article to produce and are generally acceptable for use on babies and persons who are sick or otherwise confined to a bed. A conventional diaper typically is fitted to the wearer by first placing a portion of the diaper under the wearer (generally, the back portion of the diaper is placed under the buttocks and rear waist of the wearer) and then pulling the remainder of the diaper between the wearer's legs. The rear portion of the diaper then is attached to the front portion of the diaper on each side of the wearer by a connection mechanism (tab containing tape or a hook/loop fastener).

Introducing a degree of stretchability into personal care products such as diapers, sanitary napkins, adult incontinent products and the like is believed to provide these products with certain benefits. For example, U.S. Pat. No. 2,866,459 describes a stretchable, reusable cotton diaper. Stretchability is imparted to the cotton fabric by the presence of longitudinal and transverse elastic stitching. U.S. Pat. No. 3,371,668 discloses a sanitary napkin employing a nonwoven fabric with machine direction elasticity as a cover. The described fabric comprises a nonwoven web of fibers having a plurality of elastic means, in extended condition, secured to the web.

U.S. Pat. No. 4,606,964 describes a bulked web composite and method of making the same, where the composite is formed by adhering a reticulated web of elastic material to at least one gatherable web. Upon release of the tensioning forces, the gatherable web is gathered by the reticulated web to form a bulked web composite. U.S. Pat. No. 4,847,134 discloses a stretchable absorbent undergarment, including an inner layer or topsheet, a liquid impervious outer layer or backsheet, an absorbent layer and a stretchable layer. The stretchable layer is positioned between the topsheet and the backsheet such that the stretchable layer is exposed to the exudates.

U.S. Pat. No. 5,957,908 discloses an elastomeric ear panel used with “convertible” absorbent articles, whereby the ear panels (or flaps) are said to enable the use of the article as a conventional diaper, or as a “pull-on” diaper. The specifically described ear panels impart the stretchability characteristics described therein. U.S. Pat. No. 5,938,652 discloses an absorbent article having a selectively elasticized waist flap that forms a pouch to contain the body exudates. The specific waist flap described therein is said to conform better to the wearer's back during use, while the remainder of the absorbent article is positioned away from the wearer's back, thus forming a pouch.

U.S. Pat. No. 5,451,219 discloses a stretchable absorbent article whereby the liquid permeable topsheet is connected to the liquid impermeable backsheet in discrete and non-discrete regions thereby forming a series of longitudinal pleats having channels disposed therein. The absorbent article is described as capable of absorbing a desirable amount of liquids, and at the same time capable of being elastically stretchable and contractible to better conform to the wearer's body during use. U.S. Pat. No. 5,411,498 discloses a stretchable shaped absorbent garment containing at least two generally parallel elastomeric strands stretch bonded to the garment along its length. When the article is in a relaxed state, the elastomeric strands are reduced in length rendering the article stretchable.

The disclosures of all of the aforementioned United States patents are incorporated by reference herein in their entirety. Specifically, various elements, absorbent article configurations, and the like, are useful in the context of the present invention, as will be readily understood by one having ordinary skill in the art upon reading the remaining disclosure herein.

### SUMMARY OF THE INVENTION

It is a feature of embodiments of the invention to provide absorbent articles that are more comfortable to the user, and that can adapt and stretch to fit more users.

In accordance with these and other features of the invention, there is provided an absorbent article including a chassis having a chassis layer with a chassis surface area, an elastic portion having a first carrier layer with a first surface area, and an elastic layer attached to the first carrier layer and having an elastic layer surface area; a topsheet attached to the chassis, at least a portion of the topsheet being liquid permeable; and an absorbent material disposed between the topsheet and the chassis, wherein the elastic layer is an elastic scrim.

In accordance with other embodiments of the invention, a method of making an absorbent article is provided. The method includes feeding a chassis layer onto a surface; feeding an elastic portion onto the chassis layer, the elastic portion having a carrier layer and an elastic layer, the elastic layer being an elastic scrim; bonding the elastic portion to the chassis layer; applying an absorbent material to one of the chassis layer and the elastic portion; and applying a topsheet to one of the chassis layer and the elastic portion such that the absorbent material is between the chassis layer and the top sheet.

These and other features of the invention will be readily apparent to those skilled in the art upon reading this disclosure in connection with the attached drawing figures.

### BRIEF DESCRIPTION OF THE DRAWINGS

FIG. 1 is a partial exploded view of an example of an embodiment of the invention;

FIG. 2 is a plan view of an example of an absorbent article in accordance with the invention;

FIG. 3 is an exploded isometric view of an example of an absorbent article chassis in accordance with an embodiment of the invention;

FIG. 4 is an exploded isometric view of an example of an absorbent article chassis in accordance with another embodiment of the invention;

FIG. 5 is a plan view of an example of an absorbent article in accordance with the invention;

FIG. 6 is a plan view of an example of an absorbent article in accordance with the invention;

FIG. 7 is a plan view of an example of a method of making an absorbent article in accordance with the invention;

FIG. 8 is a plan view of an example of a method of making an absorbent article in accordance with the invention;

FIG. 9 is an isometric partial cut-away view of an example of an embodiment of the invention;

FIG. 10 is a view of a pant style example of an embodiment of the invention; and

FIG. 11 is a side view of an example of a method of the invention.

### DETAILED DESCRIPTION OF PREFERRED EMBODIMENTS

“Garment,” as used herein, refers to articles and garments that absorb and contain body exudates, and more specifically refers to articles and garments that are placed against or in proximity to the body of the wearer to absorb and contain the various exudates discharged from the user's body. A non-exhaustive list of examples of “absorbent articles” and garments includes diapers, diaper covers, disposable diapers, training pants, feminine hygiene products, and adult incontinence products. The invention can be used with all of the foregoing classes of absorbent articles and garments, without limitation, whether disposable or otherwise. Furthermore, the invention will be understood to encompass, without limitation, all classes and types of absorbent articles and garments, including those described above.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

The term “component” can refer, but is not limited, to designated selected regions, such as edges, corners, sides or the like; structural members, such as elastic strips, absorbent pads, stretchable layers or panels, layers of material, or the like; or a graphic. The term “graphic” can refer, but is not limited, to any design, pattern, indicia or the like.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “topsheet” and “backsheet” denote the relationship of these materials or layers with respect to the absorbent core. It is understood that additional layers may be present between the absorbent core and the topsheet and backsheet, and that additional layers and other materials may be present on the side opposite the absorbent core from either the topsheet or the backsheet.

Absorbent garments and diapers may have a number of different constructions and configurations. In each of these, it generally is the case that an absorbent core is disposed between a liquid pervious, body-facing topsheet, and a liquid impervious, exterior facing backsheet. In some cases, one or both of the topsheet and backsheet may be shaped to form a pant-like garment. In other cases, the topsheet, backsheet and absorbent core may be formed as a discrete assembly that is placed on a main chassis and the chassis is shaped to form a pant-like garment. The garment may be provided to the consumer in the fully assembled pant-like shape, or may be partially pant-like and require the consumer to take the final steps necessary to form the final pant-like shape. In the case of training pant-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure.

Although various embodiments of the invention are described in the context of a diaper, it is readily apparent and understood that this is not intended to limit the invention. The present invention may be used with any other absorbent garment having elastics incorporated therein.

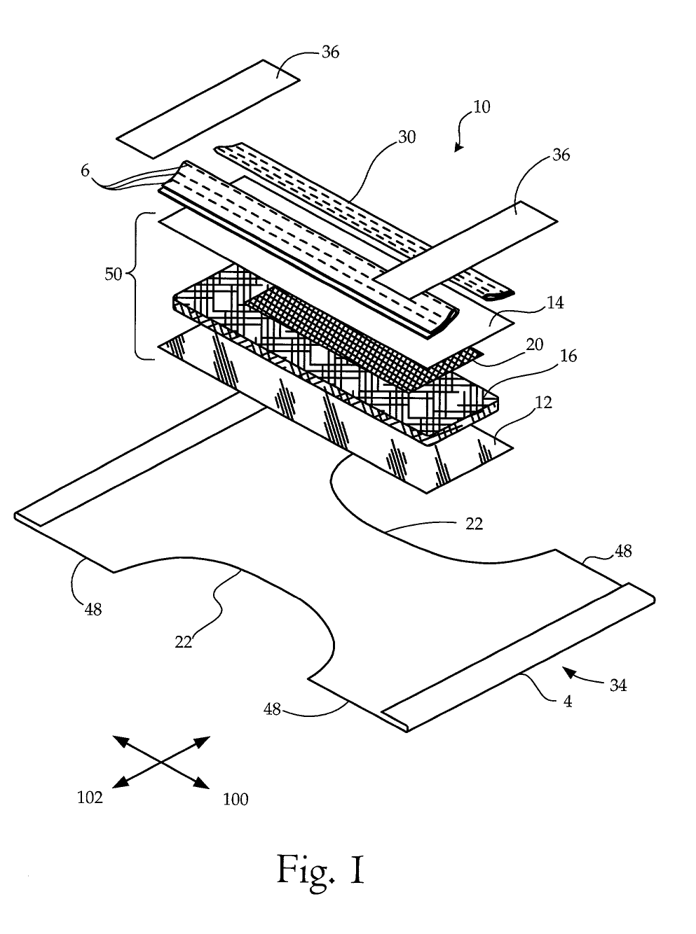

The present invention is described generally with reference to FIG. 1. FIG. 1 is an exploded view of an embodiment of the present invention with the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer.

In the embodiment of FIG. 1, the garment 10 preferably comprises a main chassis 34 that forms a pant-like garment 10 having two leg hole cutouts 22 and longitudinal ends 4. The pant-like structure may be formed by joining lateral edge portions 48 to one another to form side seams. The lateral edge portions 48 may be joined during manufacture by any manner known in the art or combinations thereof. Examples of suitable joining mechanisms include: adhesives such as hot melt adhesives and construction adhesives, chemical or solvent bonding, stitching, heat bonding, autogenous bonding, and, preferably, ultrasonic welding. The lateral edge portions 48 also may be joined by a user with the assistance of adhesive strips or mechanical fasteners (not shown). When the lateral edge portions 48 are joined, leg hole cutouts 22 along the lateral edges of the garment 10 form leg holes, and the longitudinal ends 4 of the garment 10 form a waist encircling edge.

A core assembly 50 preferably is disposed on the interior of the chassis 34. The core assembly 50 may comprise an absorbent core 16 disposed between an exterior facing moisture impervious barrier film 12 or “backsheet,” and a moisture pervious body-contacting inner layer 14 or “topsheet.” Each of the backsheet 12, topsheet 14 and absorbent core 16 may comprise a plurality of layers of materials. In the embodiment depicted in FIG. 1, the backsheet 12, topsheet 14, and absorbent core 16 comprise a subassembly that may be attached to the chassis 34. It should be readily apparent that in other embodiments one or both of the topsheet 14 and backsheet 12 may be shaped to form the main body of a pant-like garment thereby eliminating the need for a separate chassis 34. In still another embodiment of the invention, the backsheet 12, topsheet 14, and absorbent core 16 may be assembled and used without ever being shaped as a pant-like garment, such as when used as a feminine care product.

The chassis 34 may comprise a nonwoven polyethylene or polypropylene or any other suitable garment material known in the art or hereafter discovered. All or part of the chassis 34 may comprise a liquid pervious or liquid impervious material or may be zone-treated to be partially liquid pervious or impervious. The composition of chassis 34 will be discussed in more detail below.

The backsheet 12 may comprise a laminate of multiple layers of materials that have similar or different properties. The backsheet 12 preferably is made from a substantially liquid impervious material. The selection and manufacture of such materials is well known in the art, and is disclosed, for example, in U.S. Pat. No. 6,123,694 issued to Peniak et al., and U.S. Pat. No. 6,176,952 issued to Maugans et al., each of which is incorporated herein by reference in its entirety, and in a manner consistent with the present invention. In one embodiment, the backsheet 12 is made from a thin thermoplastic material, such as a pigmented polyethylene film having a thickness in the range of 0.02-0.04 mm. The backsheet 12 may also have a laminate construction comprising one or more layers of meltblown polypropylene or meltblown polyethylene, sandwiched between layers of spun-bonded material (often referred to as an “SMS” laminate). Additional layers may be added to the backsheet 12 or the backsheet 12 may be treated with chemicals in order to provide it with other desirable properties, such as to improve the tactile feel, or “hand.” The backsheet 12 may also be entirely or partly gas pervious to allow the garment to circulate air, or “breathe.”

The topsheet 14, which preferably overlays the backsheet 12, can be made from a substantially liquid pervious material to allow body exudates to penetrate into the absorbent core 16. The topsheet 14 may typically comprise a carded polyester fiber with a latex binder or a spun-bonded polypropylene having continuous fibers and thermally bonded by patterned calendar rolls. The topsheet 14 may be treated over all or part of its surface to render it hydrophilic, and may also be zone-treated with a surfactant to render it hydrophilic only in certain target areas. The topsheet 14 also may be treated with skin treating ingredients, such as aloe, vitamin E, and the like, which can be accomplished by a variety of methods known in the art. The topsheet 14 also may comprise an apertured material, such as an apertured film.

In an embodiment of the present invention, one or more of the topsheet 14, backsheet 12 and chassis 34 may comprise a laminate of several layers of material, which may have different physical properties. In another embodiment, one or more of the topsheet 14, backsheet 12 and chassis 34 may comprise several pieces of material, which may have dissimilar physical properties, joined at or near their edges to form a multi-paneled sheet. Such an embodiment is disclosed, for example, in U.S. Pat. No. 5,275,590 issued to Huffman et al., which is incorporated herein by reference in its entirety, and in a manner consistent with the present invention.

In a preferred embodiment of the invention, the topsheet 14 and chassis 34 are comprised of a nonwoven material. The topsheet 14 and backsheet 12 may also be made, however, from any other suitable material. In various embodiments, one or more of the topsheet 14, backsheet 12 and chassis 34 may be selected to provide particular benefits to the garment 10. For example, they may be selected to provide a good tactile impression, or “hand,” a comfortable fit, or gas permeability to improve the breathability of the garment 10.

The absorbent core 16 may be made from any absorbent material or materials known in the art. In one embodiment of the invention, the absorbent core 16 comprises wood fibers or other fibers such as tow fibers, chemical wood pulp, fibrous absorbent gelling material, or any other suitable liquid absorbing material, such as commercially available fluff pulp or fluffed bleached kraft softwood pulp or fibrous absorbent gelling material. In another embodiment of the invention, the absorbent core 16 comprises a combination of a porous fibrous web and super absorbent particles. Absorbent cores are known in the art and are disclosed, for example, in U.S. Pat. Nos. 5,281,207 and 6,068,620 issued to Chmielewski et al., U.S. Pat. No. 4,610,678 issued to Weisman et. al., U.S. Pat. No. 5,137,537 issued to Herron et. al., and U.S. Pat. No. 5,147,345 issued to Young et. al., which are incorporated herein by reference in their entirety, and in a manner consistent with the present invention. In such an embodiment, the absorbent core 16 may be surrounded by a liquid pervious tissue over-wrap (not shown), or other material.

The absorbent core 16 generally is elongated along the longitudinal axis 100 of the garment, and may extend along either or both of the lateral and longitudinal axes 102, 100 to the outer perimeter of the garment. In the embodiment depicted in FIG. 1, the absorbent core 16 is substantially rectangular in shape, however, it may also have rounded ends or other shapes, such as an “I” shape or a “T” shape. The absorbent core 16 also may have channels, grooves or pockets, and may have a varying thickness.

The various parts of the garment 10 preferably are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, autogenous bonding, or any other method of affixation known or hereafter discovered. U.S. Pat. No. 4,919,738 issued to Ball et. al. discloses a method of autogenous bonding, and its disclosure is herein incorporated by reference in its entirety in a manner consistent with the invention. All of the parts may be joined to each adjacent part, but some parts may not be joined to others. In one embodiment, the topsheet 14 and backsheet 12 are bonded to one another around their perimeter regions, thereby encasing and holding the absorbent core 16 in place without having to directly join the absorbent core 16 to any other component parts of the garment 10. The topsheet 14 or backsheet 12 may also be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining means that maintains the structural integrity of the garment 10 for the duration of its use.

In the embodiment of the invention depicted in FIG. 1, the garment 10 further comprises various mechanisms for improving the garment's ability to contain body exudates, such as standing leg gathers 30. Standing leg gathers 30 may be formed by incorporating a plurality of gather elastics 6 into folds in the topsheet 14 or into additional ribbons that are attached to the garment near the leg hole cutouts 22. The gather elastics 6 cause the standing leg gathers 30 to rise above the interior surface of the garment 10, thereby forming vertical curtains of material that help contain exudates. The ribbons may be liquid pervious or liquid impervious, and more than one set of standing leg gathers 30 may be provided. The standing leg gathers 30 may be attached to the topsheet 14, backsheet 12, chassis 34 or any other suitable part of the garment such that they block or impede the passage of fluids and other exudates. Additional elastics (not shown) may also be incorporated into the chassis 34, topsheet 14 or backsheet 12 adjacent the leg holes to form conventional (i.e., non-standing) leg gathers, as is known in the art. Conventional gathers contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of each of these patents are hereby incorporated by reference in their entirety.

The core assembly 50 may comprise additional layers of material that may reduce rewet of the topsheet 14, reduce strikethrough times or otherwise improve the absorbency, dryness and other properties of the garment 10. For example, a transfer layer 20 comprising an apertured film, a foam material or an air-bonded carded, bicomponent fiber nonwoven, having a basis weight of about 40 g/m² may be disposed between the topsheet 14 and the absorbent core 16. Such multiple layer absorbent cores are known in the art and disclosed in U.S. Pat. No. 5,439,458 issued to Noel et al., which is incorporated herein by reference in its entirety, and in a manner consistent with the present invention.

The core assembly 50 may be attached to the chassis 34 by any manner known in the art, such as by ultrasonic bonding or by the use of lines of hot melt adhesive. The bond between the core assembly 50 and the chassis 34 may be reinforced by laterally-extending end strips 36 that are applied over the longitudinal ends of the core assembly 50 and bonded to the underlying structure of the garment 10. The end strips 36 also may hold the ends of the standing leg gathers 30. Such end strips 36 preferably comprise a fluid pervious nonwoven material, but may be fluid impervious or a material other than a nonwoven material. Such materials are known in the art. The end strips 36 also may help prevent the longitudinal flow of exudates past the ends of the core assembly 50, particularly if the edges of the nonwoven strips overlying the core assembly 50 are left unbonded so that they form pockets to hold exudates.

In other embodiments, adjustment strips (not shown) may be disposed on and partially attached to the garment to provide for an adjustable fit. Absorbent garments often loosen during use for various reasons, such as inelastic stretching of the various components, changes in user size, and increased loading caused by the release of body exudates into the garment 10. The adjustment strips may be formed such that they may be releasably attached to the garment 10 to reduce the circumference of the waist encircling edge, and may comprise any fastening mechanism known in the art or later discovered.

It often is desirable for an absorbent garment to contract around various parts of the wearer's body to provide improved comfort and exudate containment. In addition to the standing leg gathers 30 or conventional gathers, and the composition of chassis 34, discussed below, waist elastics and tummy elastics may be incorporated into the garment 10 to contract the garment 10 about the wearer's waist and stomach. Such elastics are typically stretched as they are joined to the garment 10 so that the contraction of the elastics causes the garment 10 to contract about the wearer. The elastics may also be applied in an unstretched state then mechanically stretched to create an elasticized region in the garment (often called a zero-strain laminate). The elastics also may be applied in an inelastic state then heat activated to cause them to be come elasticized. The elastics may be made from natural or synthetic rubber, elastomers, LYCRA® elastomer (available from E. I. DuPunt de Nemours and Company, a business having offices in Wilmington, Del.), polyurethane, heat shrinkable polymer ribbons, or any other suitable elastic material or composite.

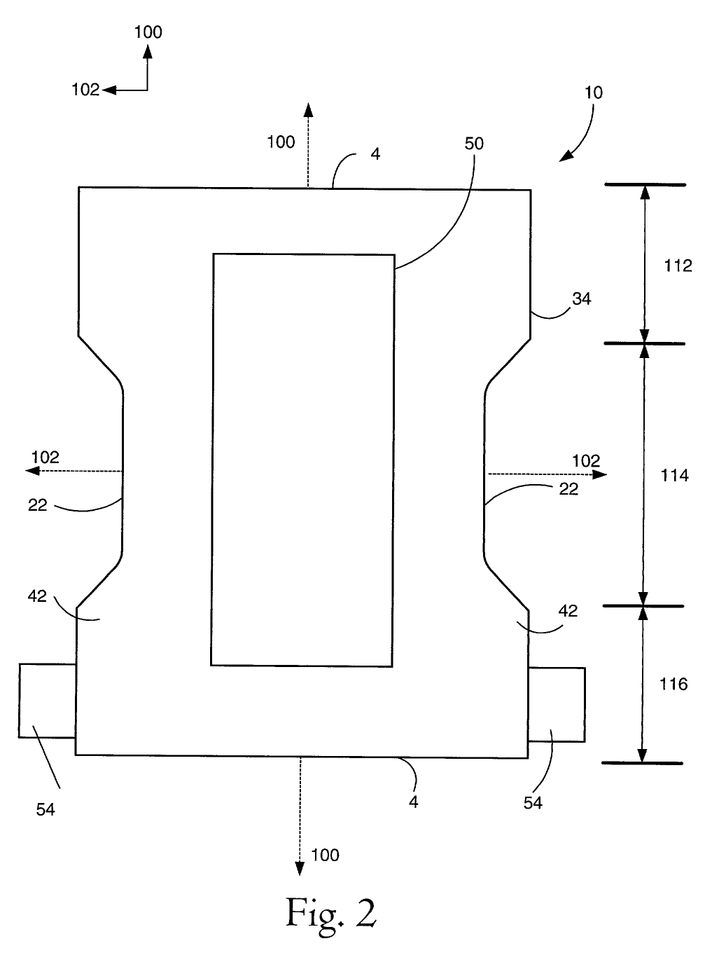

FIG. 2 shows a partial plan view of an example of an embodiment of the invention in its flat, uncontracted state (i.e. with elastic induced contraction pulled out).

Article 10 has a front waist section 112, a back waist section 116 and a crotch section 114 positioned between front waist section 112 and back waist section 116. The lines of demarcation between front waist section 112, crotch section 114, and back section 116 show just one example of the relative sizes of these sections. Chassis 34 has a chassis inside surface 42, longitudinal ends 4 and leg hole cutouts 22. The core assembly 50 is located on chassis inside surface 42. The purpose of core assembly 50 is to absorb body exudates produced by the user.

In use, the embodiment shown in FIG. 2 is wrapped around the user such that one leg hole cutout 22 is adjacent the user's left leg, the other leg hole cutout 22 is adjacent the user's right leg, one longitudinal end 4 is adjacent the user's back at his waist and the other longitudinal end 4 is adjacent the user's abdomen. Embodiments such as that shown in FIG. 2 are preferably provided with some sort of attachment device such as, for example, tabs 54. Tabs 54 can be, for example, covered with an adhesive or one part of a hook and loop fastener.

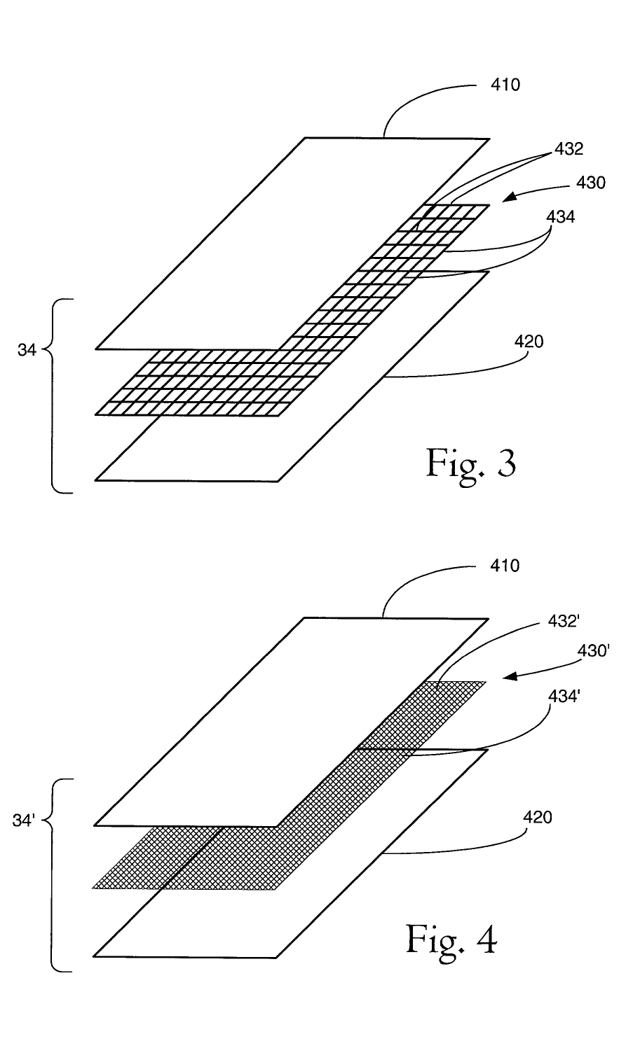

FIGS. 3 and 4 show examples of the composition of chassis 34 shown in FIG. 2. In FIG. 3, chassis 34 has a first chassis layer 410 and a second chassis layer 420 with an elastic layer 430 positioned there between. Elastic layer 430 in this example is an elastic scrim having first dimensional scrim members 432 and second dimensional scrim members 434 intersecting first dimensional scrim members 432 at right angles. When chassis 34 is assembled, elastic layer 430 is stretched while first chassis layer 410, second chassis layer 420 and elastic layer 430 are bonded together. In some embodiments, first chassis layer 410 is bonded only to second chassis layer 420, allowing scrim members 432, 434 to move relative to first chassis layer 410 and second chassis layer 420. In other embodiments, first chassis layer 410 and second chassis layer 420 are bonded to specific portions of elastic layer 430 such that, for example, only first dimensional scrim members 432 are free to move relative to first chassis layer 410 and second layer 420 while second dimensional scrim members 434 are fixed relative to first chassis layer 410 and second chassis layer 420. In other embodiments, first chassis layer 410, second chassis layer 420 and elastic layer 430 are bonded together such that no portion of elastic layer 430 is free to move relative to first chassis layer 410 or second chassis layer 420.

In some embodiments, both first dimensional scrim members 432 and second dimensional scrim members 434 are elastic. In other embodiments, only first dimensional scrim members 432 or second dimensional scrim members 434 are elastic. By selectively using elastic and non-elastic scrim members and/or selectively bonding particular scrim members to first chassis layer 410 and/or second chassis layer 420, the gathering characteristics of article 10 can be varied throughout the article.

FIG. 4 shows an example of chassis 34′ in which elastic layer 430′ has first dimensional scrim members 432′ and second dimensional scrim members 434′. Second dimensional scrim members 434′ intersect with first dimensional scrim members 432′ at an angle other than 90 degrees. The specific angle between first dimensional scrim members 432′ and second dimensional scrim members 434′ shown in FIG. 4 is merely an example and any angle can be used. By altering the angle between scrim members, different gathering properties of chassis 34′ can be achieved. This variable used in conjunction with the bonding and the elastic variables discussed above provide tremendous flexibility in designing the elastic and gathering properties of article 10.

While FIGS. 3 and 4 show elastic layers having uniform scrim member orientation, it is noted that within a particular chassis 34 the characteristics of elastic chassis layer 430 can vary so as to provide scrim members having stronger elastic pull and/or varying angular placement at different locations within chassis 34. In preferred embodiments, elastic layer 430 is designed to use scrim members having different elastic strengths and different angular orientations as well as being selectively bonded to first chassis layer 410 and/or second chassis layer 420.

In preferred embodiments, chassis 34 is liquid impervious so as to prevent body exudates from escaping through article 10.

The elastic layer 430 in the example shown in FIG. 2 has a surface area substantially equal to the surface are of chassis 34. That is, elastic layer 430 is present at substantially all areas of chassis 34.

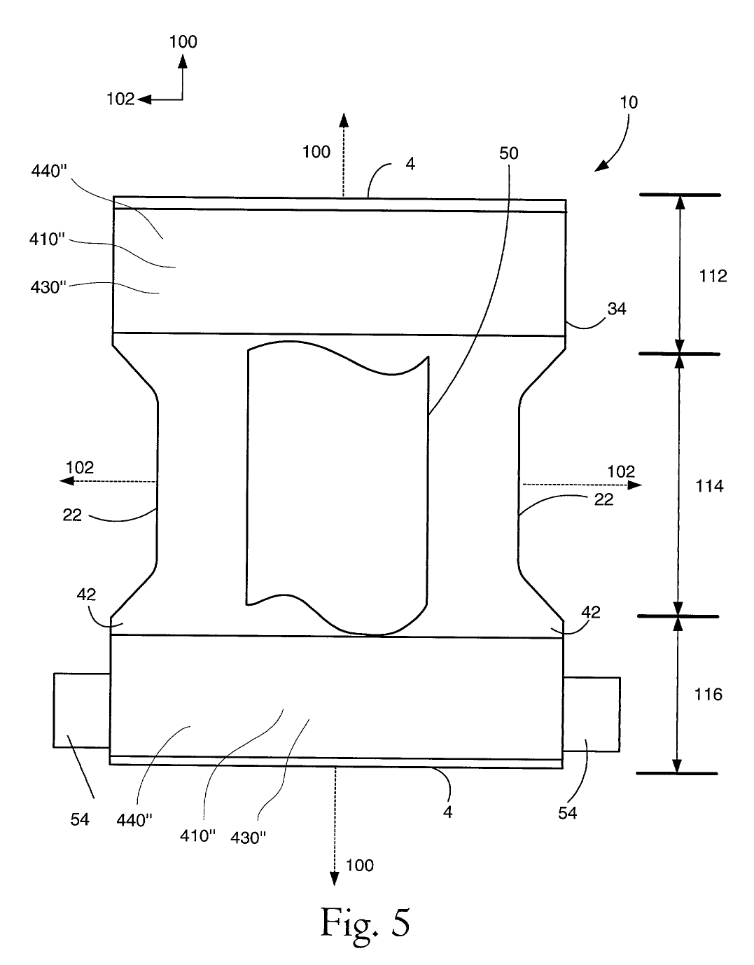

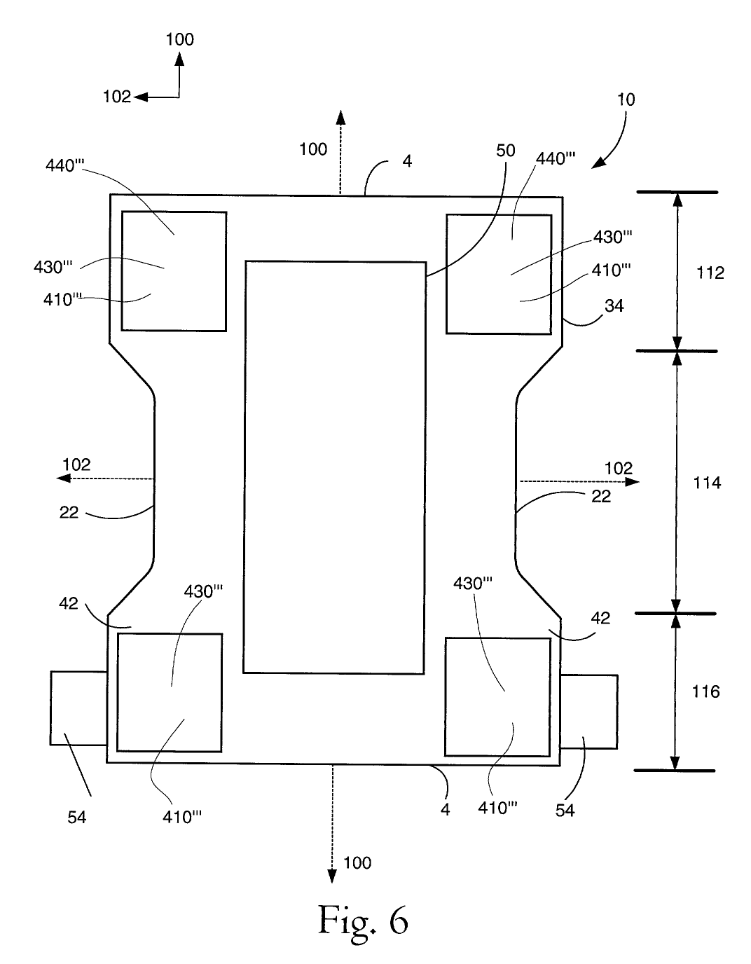

In other embodiments, the elastic layer has a surface area substantially less than the surface area of chassis 34. In FIG. 5, elastic layers 430″ are in front waist section 112 and back waist section 116 only. In these examples, elastic layers 430″ are each bonded to a first carrier layer 410″ to form elastic portions 440″ that are individually applied to chassis 34. In FIG. 5, core assembly 50 is shown cut away for clarity. FIG. 6 shows another example of an article having a plurality of elastic portions 440′″. In FIG. 6, there are four elastic portions 440′″, each having a first carrier layer 410′″ and an elastic layer 430′″. Each elastic portion 440′″ in FIG. 6 is located at a side region of either front waist section 112 or back waist section 116 to provide desirable elasticity at the side waist areas of a wearer of the article.

While the elastic portions shown in FIGS. 5 and 6 have been described as having only a first carrier layer, it is noted that the elastic portions can also have a second carrier layer. In some embodiments, the elastic layer is sandwiched between the first and second carrier layers. Similarly, while the examples shown in FIGS. 3 and 4 are described as having first and second chassis layers, it is noted that some embodiments have only one chassis layer.

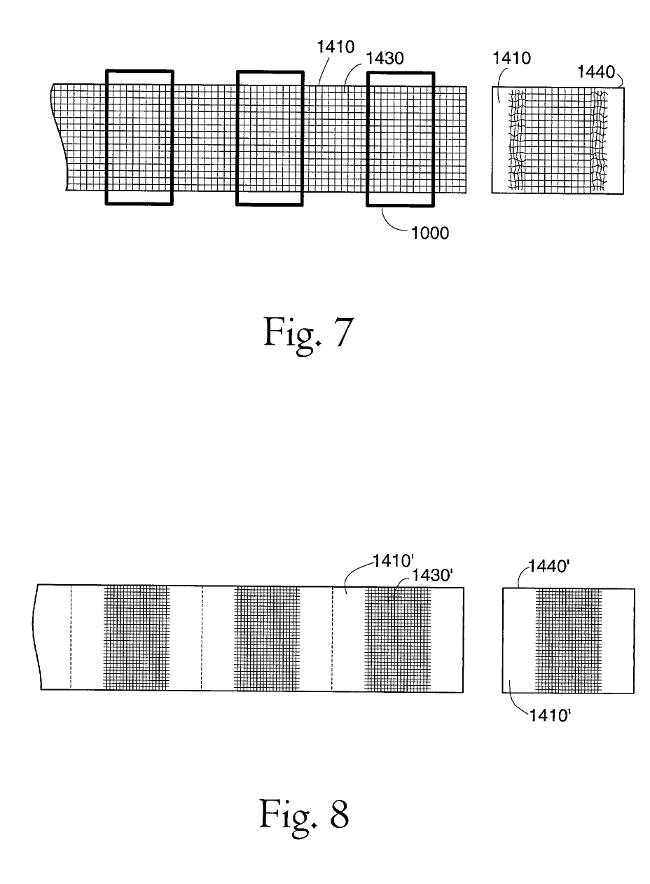

While FIGS. 3 and 4 show elastic layer 430, 430′ as having a surface area substantially equal to the surface area of the chassis layers, it is noted that in some embodiments, the elastic layer has a surface area smaller than the surface area of the chassis layer or carrier layer. For example, FIG. 7 shows an elastic portion 1440 having a carrier layer 1410 and an elastic layer 1430. During fabrication of particular embodiments of elastic portion 1440, a web of elastic layer 1430 is stretched and positioned above a web of carrier layer 1410 and then attached to carrier layer 1410 only within attachment region 1000. Elastic layer 1430 can be attached to carrier layer 1410 by, for example, an adhesive. When individual elastic portions 1440 are cut from the web, the portions of elastic layer 1430 that are not attached to carrier layer 1410 (and, therefore, not held in place relative to carrier layer 1410) return to their unstretched position as shown in FIG. 7. The individual elastic portions 1440 can then be attached to the chassis of the article in whatever position and orientation that is appropriate for the particular application.

FIG. 8 shows an elastic portion 1440′ having a carrier layer 1410′ and an elastic layer 1430′. During fabrication of particular embodiments of elastic portion 1440′, individual pieces of elastic layer 1430 are positioned (either in a stretched or an unstretched condition) above a web of carrier layer 1410′ and then attached to carrier layer 1410′. Elastic layer 1430′ can be attached to carrier layer 1410′ by, for example, an adhesive. The individual elastic portions 1440′ can then be attached to the chassis of the article in whatever position and orientation that is appropriate for the particular application.

While the embodiments shown in FIGS. 7 and 8 have been described with only one carrier layer 1410, 1410′, it is noted that two or more carrier layers can also be used. In particular embodiments, the elastic layer 1430, 1430′ is positioned between two carrier layers.

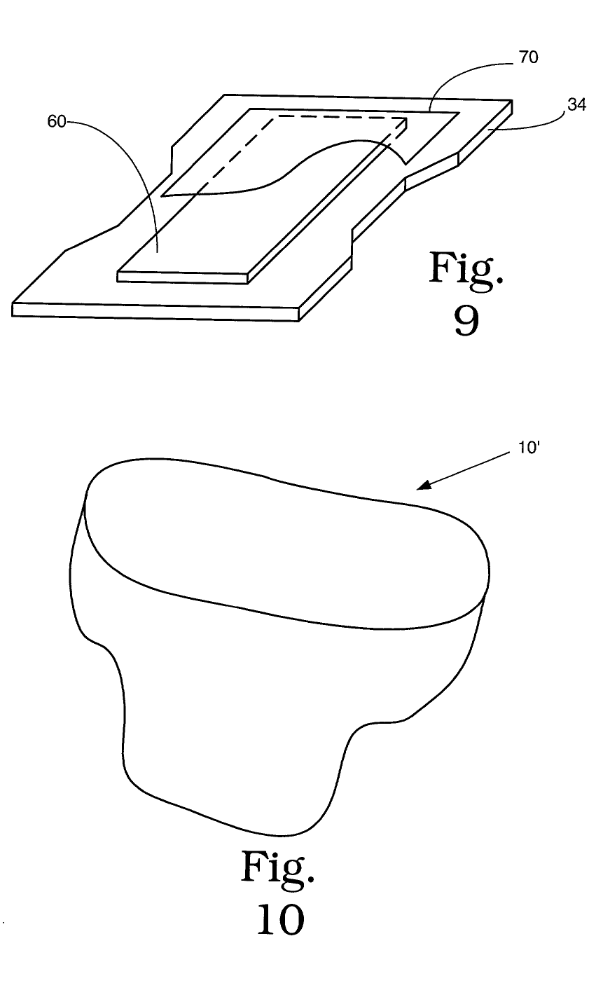

FIG. 9 shows an example of the invention having absorbent layer 60 trapped between chassis 34 and a topsheet 70. In this example, topsheet 70 would be at least partially liquid permeable so that body exudates penetrate topsheet 70 and are absorbed by absorbent layer 60. In FIG. 9 topsheet 70 is shown cut away for clarity and is shown smaller than the overall dimension of chassis 34. In addition, absorbent layer 60 is shown in FIG. 9 as rectangular in shape. Various different sizes and shapes of absorbent layer 60 can be used to provide the desired absorbency and fit. In addition, topsheet 70 is sized appropriately to cover absorbent layer 60.

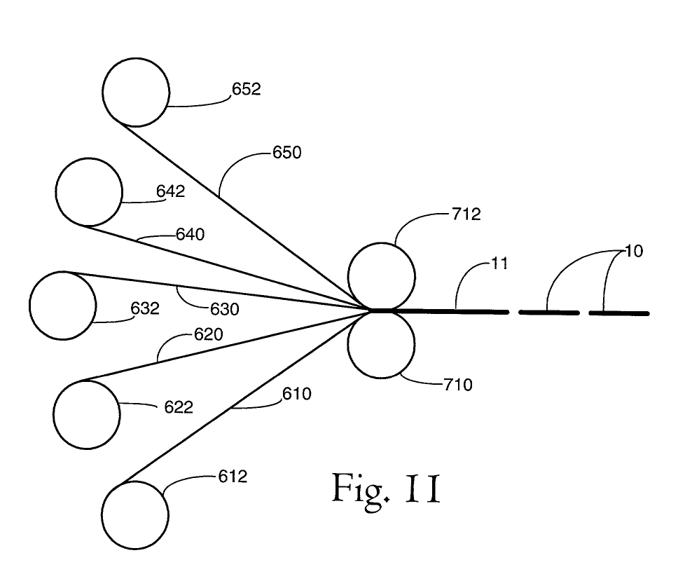

FIGS. 2-9 have used a diaper-type article as an example. FIG. 10 shows a pant-type article 10′, such as a training pant. The above description using FIGS. 2-9 as an example also applies to a pant-type article such as 10′ shown in FIG. 10. Application of the invention to a pant-type article can result in the article fitting more like normal underwear than a traditional incontinence article.

FIG. 11 shows a simplified schematic view of a method of assembling an embodiment of the invention. In FIG. 11, first element 610, for example second chassis layer 420, is fed from a first spool 612 to rollers 710 and 712. Similarly, second element 620 (for example elastic layer 430), third element 630 (for example first chassis layer 410), fourth element 640 (for example absorbent layer 60), and fifth element 650 (for example topsheet 70), are fed from spools 622, 632, 642 and 652, respectively. After the various elements are fed through rollers 710 and 712, a composite 11 is formed. Composite 11 is then cut into a predetermined shape to produce article 10. In preferred embodiments, at least elastic layer 430 would be stretched in at least one dimension during assembly.

While FIG. 11 shows the assembly of five elements, it is noted that any number of elements can be assembled in this or other manners. For example, the three elements of the examples of chassis 34 described above can be assembled using the method shown in FIG. 11 while other elements of article 10 are added at different points in the assembly line. Other methods of assembling known in the art can also be applied to assembling articles of the invention.

While the invention has been described with reference to particularly preferred embodiments and examples, those skilled in the art will appreciate that various modifications may be made thereto without significantly departing from the spirit and scope of the invention.

We claim:

1. An absorbent article, comprising:
a chassis having a chassis layer with a chassis surface area;
an elastic portion having
a first carrier layer with a first surface area, and
an elastic layer attached to the first carrier layer and having an elastic layer surface area;
a topsheet attached to the chassis, at least a portion of the topsheet being liquid permeable; and
an absorbent material disposed between the topsheet and the chassis,
wherein the elastic layer is an elastic scrim.

2. The absorbent article of claim 1, wherein the elastic layer is unidirectionally elastic.

3. The absorbent article of claim 1, wherein the elastic layer is bidirectionally elastic.

4. The absorbent article of claim 1, wherein the elastic layer is elastic in more than two directions.

5. The absorbent article of claim 1, wherein the elastic layer surface area is substantially equal to the chassis surface area.

6. The absorbent article of claim 1, wherein the elastic portion comprises a plurality of elastic portions,
each of the plurality of elastic portions having a surface area less than the chassis surface area.

7. The absorbent article of claim 6, wherein a total surface area of the plurality of elastic portions is less than the chassis surface area.

8. The absorbent article of claim 1, wherein the chassis further comprises
a front waist region;
a back waist region; and
a crotch region located between the front and back waist regions such that when worn by a user the front and back waist regions form a loop around the user's waist.

9. The absorbent article of claim 8, wherein the article is a pant style article.

10. The absorbent article of claim 9, further comprising an irreversibly destructible connection that connects the front waist region to the back waist region.

11. The absorbent article of claim 8, wherein the elastic portion comprises a first strip,
the first strip being located in one of the front waist region and the back waist region.

12. The absorbent article of claim 11, wherein the elastic portion further comprises a second strip,
the second strip being located in one of the front waist region and the back waist region.

13. The absorbent article of claim 1, wherein the scrim is substantially completely bonded to one of the first and second chassis layers.

14. The absorbent article of claim 1, wherein the scrim is bonded to one of the first and second chassis layers only at predetermined portions of the scrim, the predetermined portions being less than the entire scrim.

15. The absorbent article of claim 1, wherein the scrim comprises
first scrim members having a first elasticity; and
second scrim members have a second elasticity different from the first elasticity.

16. The absorbent article of claim 15, wherein the first and second scrim members are colinear.

17. The absorbent article of claim 1, wherein the article is a pant style article.

18. The absorbent article of claim 1, wherein one of the first and second chassis layers is a non-woven material.

19. The absorbent article of claim 1, wherein elastic portion further comprises a second carrier layer with a second surface area.

20. The absorbent article of claim 19, wherein the elastic layer is positioned between the first carrier layer and the second carrier layer.

21. The absorbent article of claim 20, wherein the second surface area is substantially equal to the first surface area and the elastic layer surface area.

22. A method of making an absorbent article, the method comprising:
feeding a chassis layer onto a surface;
feeding an elastic portion onto the chassis layer, the elastic portion comprising
a carrier layer, and
an elastic layer, the elastic layer being an elastic scrim;
bonding the elastic portion to the chassis layer;
applying an absorbent material to one of the chassis layer and the elastic portion; and
applying a topsheet to one of the chassis layer and the elastic portion such that the absorbent material is between the chassis layer and the top sheet.

23. The method of claim 22, wherein the elastic portion has a surface area substantially equal to a surface area of the chassis layer.

24. The method of claim 22, wherein the scrim is unidirectionally elastic.

25. The method of claim 22, wherein the scrim is bidirectionally elastic.

26. The method of claim 22, wherein the scrim is elastic in more than two directions.

27. The method of claim 22, wherein the scrim is substantially completely bonded to the chassis layer.

28. The method of claim 22, wherein the scrim is bonded to one of the chassis layer only at predetermined portions of the scrim, the predetermined portions being less than the entire scrim.

29. The method of claim 22, wherein the scrim comprises
first scrim members having a first elasticity; and
second scrim members have a second elasticity different from the first elasticity.

30. The method of claim 29, wherein the first and second scrim members are colinear.
